(19) United States
(12) Patent Application Publication
(10) Pub. No.: US 2017/0181708 A1
(43) Pub. Date: Jun. 29, 2017

(54) METHODS AND SYSTEMS FOR DETECTING PHYSIOLOGICAL PARAMETER MEASUREMENTS

(71) Applicant: LifeBEAM Technologies Ltd., Tel-Aviv (IL)

(72) Inventors: Zvi Orron, Tel-Aviv (IL); Shimon Hayun, Rosh HaAyin (IL); Yonathan Nativ, Tel-Aviv (IL); Jonathan Aprasoff, Kfar-Saba (IL)

(21) Appl. No.: 14/981,285

(22) Filed: Dec. 28, 2015

(51) Int. Cl. A61B 5/00; A61B 5/024

(52) U.S. Cl. CPC A61B 5/7214; A61B 5/02433; A61B 5/6817; A61B 5/6843; A61B 5/721; A61B 2562/0219; A61B 2562/0238

(57) ABSTRACT

System and methods for detecting one or more physiological parameters of a subject is provided. Detecting of physiological parameters can occur through a wearable device that can transmit and receive electromagnetic waves. The received electromagnetic waves are reflections from tissue of the subject. The reflections have motion and noise substantially removed. The one or more physiological parameters can be determined from motion and noise removed signals.

### FIELD OF THE INVENTION

The invention relates generally to detection of physiological parameters of a subject. More specifically, one or more physiological parameters (e.g., heart rate) are detected based on merging at least two electromagnetic reflection signals and/or reducing motion noise on the merged signal.

### BACKGROUND

In general, physiological parameter detection devices (portable heart rate monitors, thermometers, SPO2, etc.) can sense or detect a variety of physiological parameters. Current physiological parameter detection devices are typically dedicated or otherwise stand-alone devices. For example, some portable heart rate monitors can be dedicated solely to the function of measuring and determining the heart rate of the individual using the device.

Some current technologies allow for a sensor for physiological parameter detection to be integrated with or otherwise combined with wearable devices (e.g., an earbud of a headset.) For example, a light emitting diode and a photosensor can be integrated into an earbud or a watch, such that electromagnetic signals are transmitted to and reflected from a boundary of the tissue of a subject. Such devices can acquire the reflected signals, perform signal processing on the acquired reflected signals to obtain one or more physiological parameters (e.g., heart rate), and/or transmit the one or more physiological parameters to a processor capable of displaying or otherwise utilizing the physiological parameter to a user' device (e.g., a mobile phone).

In current wearable devices, the accuracy of the physiological parameter detection can be low due to, for example, motion of the subject. For example, when a subject is exercising (e.g., running, jumping, etc. . . . ) with an earbud in the ear, the motion of the subject's movement can cause the wearable device's position and the sensor therein, to move during sensing. The movement can cause a difference in position of the sensor. Physiological parameter detection can rely on the changes of optical behavior of tissue as blood pulses through it, thus changing the sensor's position can cause measurements to be at differing locations on the tissue. Taking measurements at differing locations of the tissue can cause the sensed electromagnetic signals to lose amplitude and/or frequency accuracy.

Reduced accuracy of the physiological parameter detection can also be caused by a change in ambient light. For example, when a subject is running and moves between a sunny area to a shady area, the change in ambient light can cause the sensed electromagnetic signals to lose amplitude and/or frequency accuracy.

Reduced accuracy of the physiological parameter detection can also be caused by a change in activity level of the subject. When detecting the physiological parameter of heart rate, current algorithms for determining heart rate can rely on a probabilistic determination that uses a previous heart rate in determination of the current heart rate. When a subject is running, their heart rate can be in the 150 to 180 beats per minute range, and when sitting their heart rate can be in the 60 to 100 beats per minute range. For a probabilistic determination of heart rate that relies on the previous heart rate, this rapid change in heart rate can cause inaccuracies in the probabilistic determination.

Another difficulty with current devices for physiological parameter detection is that they can require computationally intensive computer processing, which can delay results and/or quickly drain a battery. For example, in the case of heart rate detection, if the heart rate provided to the subject is delayed due to the time it takes for processing, it can result in the subject being presented with a stale heart rate value.

### SUMMARY OF THE INVENTION

One advantage of the invention is that it allows for a reduction/substantial elimination of motion noise in the detected physiological parameters, thus allowing for higher accuracy.

Another advantage of the invention is that it can allow for an improvement in overall system performance due to, for example, using the detected physiological parameters to modify future detections. For example, when a subject is resting, an intensity of transmitted light, sampling rate, and/or digital signal processing length can be at minimum values, and still provide highly accurate physiological parameters. As the subject's activity increases, the intensity of transmitted light, sampling rate, and/or digital signal processing length can be modified, to ensure that at higher activity levels the physiological parameters detected are accurate. In this manner, the invention can allow for reduction in the overall number of computations required for detections, battery life improvement and/or minimization of delay in providing the physiological parameter to the subject.

Another advantage of the invention is improved physiological parameter detection over a range of tissue tones. Another advantage of the invention is that is allows for accurate physiological parameter detection, even if the device is not perfectly positioned on the body.

Another advantage of the invention is that it allows for highly accurate detection of tissue contact between a wearable device and a subject.

Another advantage of some embodiments of the invention is that it can allow a subject to listen to audio simultaneous with physiological parameter detection, due to the ability of the wearable device to process physiological measurements and audio signals at the same time.

In one aspect, the invention features a method of detecting one or more physiological parameters. The method involves, transmitting one or more electromagnetic signals from a wearable device to a tissue boundary of a subject. The method involves, receiving at least two reflected signals based on reflections from the tissue of the one or more electromagnetic signals. The method involves, receiving a reference signal based on motion of the subject. The method involves, merging the at least two reflected signals based on a position of one or more receivers that receive the at least two reflected signals to create a merged signal. The method involves, removing at least a portion of motion noise from the merged signal based on the reference signal to obtain a noise-reduced merged signal. The method involves, determining the one or more physiological parameters based on the noise-reduced merged signal.

In some embodiments, the reference signal is one or more three-dimensional acceleration signals. The some embodiments, determining the one or more physiological parameters also involves receiving acceleration signals from an accelerometer coupled to the subject, determining an activity state of the subject based on the acceleration signals, and determining a frequency for the one or more physiological parameters based on the activity state of the subject.

In some embodiments, removing at least a portion of motion noise from the merged signal also involves receiving ambient light signals from a light sensor coupled to the subject, determining a linear combination signal based on the reference signal, the ambient light signals and the merged signal, determining a non-linear transformation of the linear combination signal based on the reference signal and the ambient light signals, and applying the non-linear transformation to obtain the noise-reduced merged signal.

In some embodiments, the one or more physiological parameters include a heart rate. In some embodiments, the method also involves de-correlating the reference signal based on principle component analysis of the reference signal. In some embodiments, determining the one or more physiological parameters also involves sampling the at least two reflected signals based on one or more previously determined physiological parameters of the subject.

In some embodiments, the one or more electromagnetic signals includes a wavelength, amplitude or both that is based on tissue color of the subject, ambient light, or any combination thereof. In some embodiments, the one or more electromagnetic signals are transmitted with a wavelength of green light or infrared light.

In some embodiments, the method also involves increasing or decreasing an amplitude of the one or more electromagnetic signals based on ambient light surrounding the subject. In some embodiments, the method also involves normalizing the at least two reflected signals such that each of the at least two reflected signals have a variance below a predetermined variance threshold.

In some embodiments, the one or more electromagnetic signals are generated by one or more light emitting diodes. In some embodiments, the at least two reflected signals are received by one or more photo sensors. In some embodiments, the at least two reflected signals are photoplethysmogram (PPG) signals.

In some embodiments, determining the one or more physiological parameters also involves comparing the one or more physiological parameters to previously determined one or more physiological parameters, respectively, and for each of the one or more physiological parameters, if the comparison is greater than a predetermined threshold, discarding the respective determined one or more physiological parameters.

In some embodiments, the method involves receiving by the wearable device audio signals and processing the audio signals on a processor within the wearable device, the processor being the same processor that the one or more physiological parameters are processed on.

In some embodiments, the wearable device is an earbud, a watch, a chest strap, or any combination thereof.

In another aspect, the invention includes a system for detecting one or more physiological parameters. The system includes one or more emitters to transmit one or more electromagnetic signals from a wearable device to a tissue boundary of a subject. The system also includes one or more detectors to receive at least two reflected signals based on reflections from the tissue of the one or more electromagnetic signals. The system also includes one or more accelerometers to receive a reference signal based on motion of the subject. The system also includes one or more processor configured to merge the at least two reflected signals based on a position of one or more receivers that receive the at least two reflected signals to create a merged signal, remove at least a portion of motion noise from the merged signal based on the reference signal to obtain a noise-reduced merged signal, and determine the one or more physiological parameters based on the noise-reduced merged signal.

In another aspect, the invention involves a method of detecting when an earbud is sufficiently inserted into an ear. The method involves transmitting a first electromagnetic energy with a wavelength from the earbud. The method also involves receiving reflected electromagnetic energy by the earbud. The method also involves determining an absolute reflection energy based on the reflected electromagnetic energy. The method also involves determining a pulsation energy based on the reflected electromagnetic energy. The method also involves determining whether the earbud is sufficiently inserted into the ear based on the absolute reflection energy and the pulsation reflection energy.

In some embodiments, the method involves determining a relative reflection energy gradient based on the reflected electromagnetic energy.

### BRIEF DESCRIPTION OF THE DRAWINGS

The foregoing and other objects, features, and advantages of the present invention, as well as the invention itself, will be more fully understood from the following description of various embodiments, when read together with the accompanying drawings. It is to be understood that the figures are exemplary in nature, and are not meant to be of scale.

FIG. 1 is a schematic diagram of a subject using a wearable device capable of detecting physiological parameters, according to an illustrative embodiment of the invention.

FIG. 2 is a schematic diagram of an exemplary wearable device of an earbud, for detecting physiological parameters, according to an illustrative embodiment of the invention.

FIG. 3 is a flow diagram of a method for detecting physiological parameters, according to an illustrative embodiment of the invention.

FIG. 4 is a flow diagram of a method for detecting a wearable device is positioned sufficiently relative to a tissue boundary, according to an illustrative embodiment of the invention.

### DETAILED DESCRIPTION

Generally, a wearable device is placed within proximity of a tissue boundary of a subject (e.g., a person). The wearable device includes one or more sensors that transmit and/or receive electromagnetic signals that are impinged upon the tissue boundary of the subject. The wearable device can also include one or more accelerometers that measure acceleration of the subject.

In operation, one or more electromagnetic signals are transmitted from the wearable device to the tissue boundary of the subject. A portion of the one or more electromagnetic signals are absorbed by the tissue, and a portion of the one or more electromagnetic signals are reflected from the tissue. At least two reflected signals are received by the one or more sensors. The at least two reflected signals are merged to create one merged signal. One or more reference signals (e.g., motion signals) are sensed by the one or more accelerometers. Motion noise is substantially removed from the merged signal based on the one or more reference signals to create a noise-reduced merged signal. The noise-reduced merged signal is used to determine one or more physiological parameters of the subject.

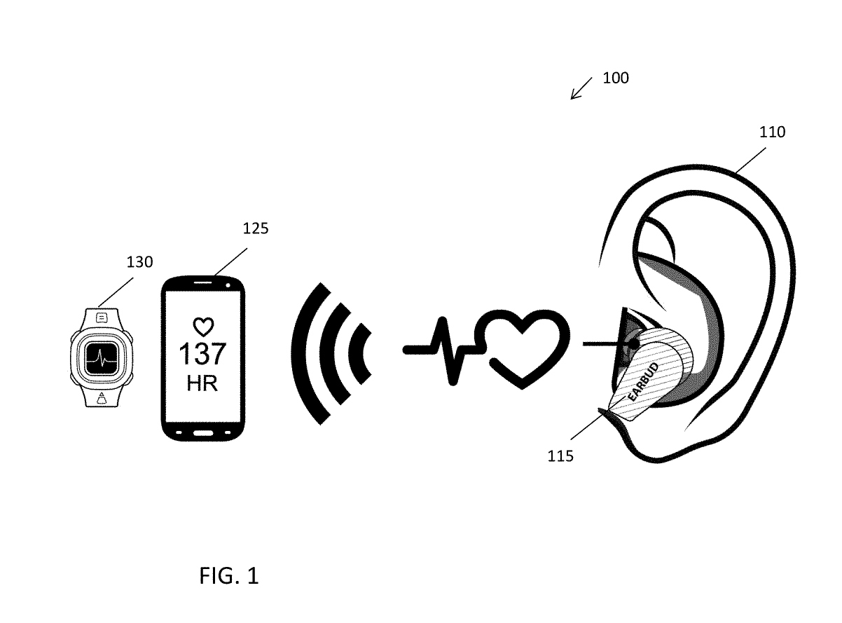

FIG. 1 is a schematic diagram 100 of a subject 110 using a wearable device 115 capable of detecting physiological parameters, according to an illustrative embodiment of the invention. The subject 110 is a human that is wearing the wearable device 115 that is an earbud (or hearing aid). The earbud can be placed on a proximal side of a tragus of an ear of the human. The human can move while wearing the wearable device.

The wearable device 115 can include one or more emitters (not shown), one or more detectors (not shown), one or more accelerometers (not shown), and one or more processors (not shown). In some embodiments, the one or more emitters, the one or more detectors, the one or more accelerometers, and/or the one or more processors can be integrated onto a single microchip. The microchip can also include an analog to digital converter to convert received electromagnetic signals from an analog format to a digital format. The microchip can also include a digital pre-processor.

The digital pre-processor can perform signal conditioning. The digital pre-processor can remove ambient noise from the received electromagnetic signals. The digital pre-processor can average multiple of the digital electromagnetic signals to remove noise. The digital pre-processor can perform filtering and/or remove a portion of motion noise.

In some embodiments, the digital pre-processor is included as part of the analog front end instead of on the microchip. In these embodiments, the microchip can include a digital interface to communicate with the analog front end.

In some embodiments, the one or more emitters are light emitting diodes. In some embodiments, the one or emitters is an laser. In some embodiments, the one or more light emitting diodes emit light in the green light spectrum. In some embodiments, the one or more light emitting diodes emit light in the infrared spectrum. In some embodiments, the infrared spectrum is emitted during a determination of whether the wearable device is placed within sufficient proximity of a tissue boundary of a subject, and when the wearable device is sufficiently positioned, the green light spectrum is emitted during detection of the one or more physiological parameters. For example, for a wearable device of an earbud, the infrared spectrum is emitted during placement of the earbud in the ear, and once the earbud is situated properly in the ear, the green light spectrum is emitted.

In some embodiments, the infrared spectrum is emitted when the subject is not moving and the green light spectrum is emitted when the subject is moving.

In various embodiments, the one or more light emitting diodes emit light in the yellow, red and/or blue range. In various embodiments, the light emitting diodes emit light in the visible light range. In some embodiments, there are two emitters.

In some embodiments, the one or more detectors are optical detectors. In some embodiments, the one or more detectors are photodiodes. In some embodiments, the one or more detectors are an array of four photodiodes. In some embodiments, the one or more detectors are phototransistors.

In some embodiments, the wearable device is 5 by 3 mm. In some embodiments, there are two detectors.

In some embodiments, the wearable device includes a wireless transmitter. The wireless transmitter can include a Bluetooth transmitter, a Wi-Fi transmitter, a cell phone transmitter, any ISM 2.4 GHz band protocol, or any combination thereof. In some embodiments, the wearable device can communicate via the wireless transmitter to a wireless network (e.g., the cloud). The wireless network can communicate with one or more computing devices (e.g., a computer of the subject, a computer in a doctor's office, a computer of a health monitoring service, etc.).

During operation, the wearable device 115 is placed in proximity of the tissue of the subject. The one or more emitters emit one or more electromagnetic signals towards the tissue of the subject. The one or more detectors receive at least two reflection signals of the one or more electromagnetic signals from the tissue of the subject. The at least two reflection signals are processed to determine whether the wearable device 115 has been placed in sufficient proximity to the tissue boundary of the subject. If the wearable device 115 has been placed in sufficient proximity to the tissue boundary of the subject, the at least two reflected signals are merged to create one merged signal. A reference signal detected by the one or more accelerometers is used to reduce/remove motion noise from the one merged signal, creating a noise-reduced merged signal. The noise-reduced merged signal is used to determine one or more physiological parameters of the subject (e.g., heart rate).

The one or more physiological parameters can be communicated to the subject. For example, for a wearable device of an earbud, the one or more physiological parameters are played on a speaker of the earbud. In some embodiments, other audio output (e.g., music, audio books, etc.) can be played to the subject via the speaker.

In some embodiments, the wearable device 115 is capable of a wired connection to another device (e.g., smartphone or computer).

For a wearable device 115 of a watch 130, the one or more physiological parameters are displayed by the watch 130 to the subject. In some embodiments, the wearable device 115 is an earbud and the one or more physiological parameters are transmitted to a watch 130 and output via a display or audio output to the subject.

In some embodiments, the one or more physiological parameters are transmitted to a smart phone 125 of the subject and output via a display or audio output to the subject. In some embodiments, the one or more physiological parameters are transmitted over a wireless network to one or more computing devices, where they are stored, displayed and/or further analyzed.

In various embodiments, the one or more processors can merge the at least two reflected signals, apply the reference signals to the merged signals, determine the one or more physiological parameters, or any combination thereof. In some embodiments, one processor controls the emitters, processes received signals (e.g., reflected signals and accelerometer signals), and controls the playing of audio output through speakers of the wearable device.

In some embodiments, the wearable device 115 includes a memory. In some embodiments, the one or more physiological parameters are stored within a memory in the wearable device 115.

In various embodiments, the processor can transmit the at least two reflected signals, the merged signal, the noise-reduced merged signal, the one or more physiological parameters, or any combination thereof, to a wireless network. In various embodiments, the wireless network includes a server that can receive the at least two reflected signals, the merged signal, the noise-reduced merged signal, the one or more physiological parameters, or any combination thereof. The server can merge the at least two reflected signals, apply the reference signals to the merged signals, determine the one or more physiological parameters, or any combination thereof.

In some embodiments, if the wearable device 115 has not been placed sufficiently near the tissue boundary of the subject an alarm issues indicating to the subject that the wearable device 115 must be repositioned.

It is apparent to one of ordinary skill in the art that the wearable device and the accompanying components shown in FIG. 1 are exemplary only and various configurations can exist (e.g., more or less user devices and/or one or more server devices in the wireless network, etc.).

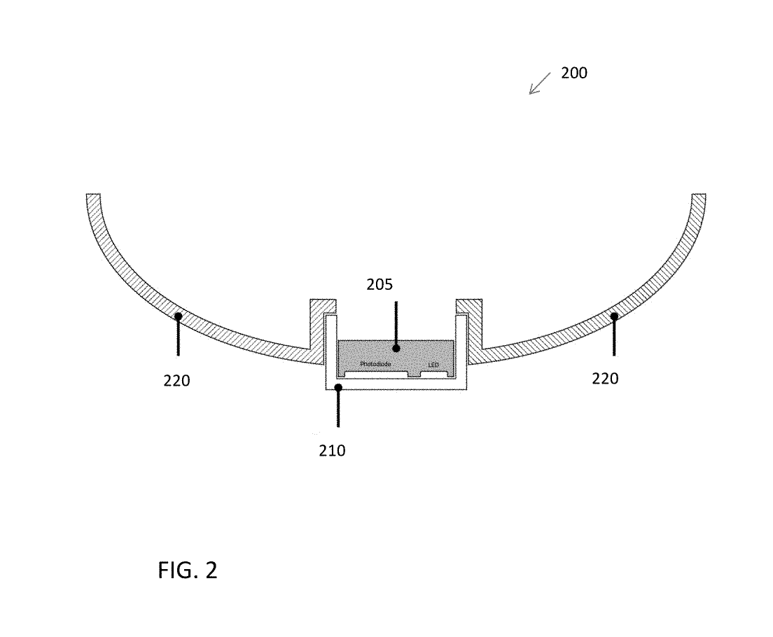

FIG. 2 is a schematic diagram of an exemplary wearable device 200 of an earbud, for detecting physiological parameters, according to an illustrative embodiment of the invention. The earbud 200 includes a sensor 205 inside of a sensor housing 210 and an earbud body 220. The sensor housing 210 can include a flat boundary portion.

The earbud body 220 is inserted into an ear of the subject. The flat boundary portion of the sensor cover is shaped to ensure that the earbud 200 can be comfortably positioned on (or as close as possible to) the desired portion of the ear. The desired portion of the car can be the Tragus, Antitragus, or the Concha of an ear.

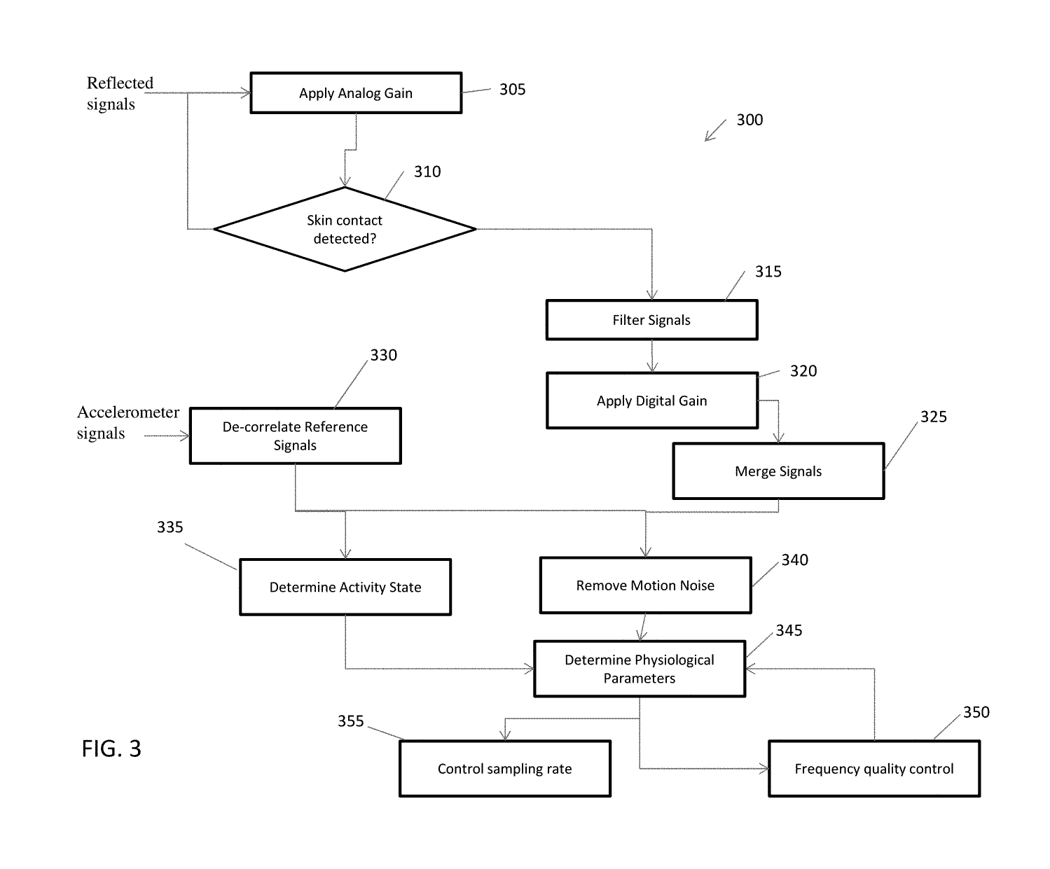

FIG. 3 is a flow diagram of a method 300 for detecting physiological parameters, according to an illustrative embodiment of the invention.

The method involves applying an analog gain to one or more transmitted electromagnetic signals (e.g., the one or more electromagnetic signals as discussed above with respect to FIG. 1) (Step 305).

The analog gain is configured to amplify each transmitted signal, reduce each transmitted signal, or keep each transmitted signal the same. The gain can be configured based on ambient light detected around the wearable device. For example, if the subject wearing the device moves from sunshine to shade, the analog gain can be configured to amplify each transmitted signal. In some embodiments, if the subject is not wearing the device, the transmitted signals can be reduced such that power can be saved. In some embodiments, the analog gain depends on skin tone of the subject. In some embodiments, the analog gain is configured to obtain a desired signal to noise ratio.

In some embodiments, the analog gain is determined based on a peak gain. The peak gain can be determined as shown below in EQN. 1:

I_{Peak}=I_{COARSE}*I_{FINE}*I_{Range} EQN. 1

Where I_{Peak} is the peak gain, I_{COARSE} is a course value that can depend on a scale range, I_{FINE} is a fine value that can depend on a fine-scale range (e.g., an integer value from 0 to 32), and I_{Range} is a sub range which is a percentage of I_{FINE}. In some embodiments, I_{COARSE} can be determined as shown below in EQN. 2

$${I}_{COARSE} = 25 + 15 * round ( \frac{scale}{2} ) \qquad EQN . 2$$

where scale is an integer value representing tone of the subject and/or contact of the wearable device with the subject. In some embodiments, the scale is determined based on Von Luschan's chromatic scale. In some embodiments, the scale is determined as shown below in Table 1.

TABLE 1

| Scale | Contact & Skin Tone |
|---|---|
| 0 | Not in contact with the skin |
| 1-5 | Very light or white, “Celtic” type |
| 6-10 | Light or light-skinned European |
| 11-15 | Light intermediate, or dark-skinned European |
| 16-21 | Dark intermediate or “olive skin” |
| 22-28 | Dark or “brown” type |
| 29-36 | Very dark or “black” type |

In some embodiments, I_{FINE} can be determined as shown below in EQN. 3

I_{FINE}=0.85+0.0125*fine_Scale EQN. 3

where fine_Scale is an integer value between 0 and 32 that can depend on a signal to noise ratio of the transmitted signal.

In some embodiments, I_{Range} can be determined as shown below in EQN. 4.

I_{Range}=0.4+0.6*n EQN. 4

where n is either 0 or 1. In some embodiments, n is based on a DC level of the transmitted signal. For example, when the DC level is saturated, n is set to 0, and when the DC level is very low, n is set to 1.

The method also involves detecting whether a wearable device (e.g., the wearable device 115 as described above in FIG. 1) is in sufficient proximity of the tissue boundary of a subject such that signal reflections can be reliable (Step 310). In some embodiments, if one of the detectors of the wearable device is within 1 mm of the tissue boundary of the subject, the wearable device is in sufficient proximity to the tissue boundary of the subject.

Whether the wearable device is in sufficient contact with the subject can be determined as shown in FIG. 6, as discussed in further detail below. If the wearable device is not in sufficient proximity to the tissue boundary of the subject, then as the detectors receive additional signals, the signals are checked until sufficient proximity to the tissue boundary of the subject is detected. If the wearable device is in sufficient contact with the tissue boundary of the subject, then the method proceeds to step 315.

The method also involves filtering two or more reflected signals (Step 315). In some embodiments, the filter is an infinite impulse response (IIR) band-pass filter. In some embodiments, the at least two reflected signals are filtered for frequencies below 0.5 hertz. In some embodiments, the at least two reflected signals are filtered for frequencies above 4.0 hertz. In some embodiments, the filter is based on the type of physiological parameter being detected.

In various embodiments, there are 2-N reflected signals, where N is an integer.

The method also involves applying a digital gain to the filtered at least two reflected signals (Step 320). The digital gain can be based on filtering and decimation of a sampling rate. The digital gain can be based on a desired signal to noise ratio. In some embodiments, the digital gain is determined as shown below in EQN. 5.

PDg=E₁^{B}PDx Taking the 8^{th} sample EQN. 5

where PDs is the summed value of the last eight reflected values and PD(n) is a vector of the reelected values.

The method also involves merging the at least two reflected signal (Step 325) resulting in a merged signal. There can be 2-N reflected signals, where N is a whole integer number. In various embodiments, N is based on the number of emitters, number or detectors, or any combination thereof. In some embodiments where the one or more physiological parameters being detected is heart rate, there are 2-N photoplethysmogram (PPG) signals.

In some embodiments, merging the at least two reflected signals is based on independent component analysis. In some embodiments, merging the at least two reflected signals involves reducing motion artifacts in the merged signal. In these embodiments, the quasi-periodicity of the 2-N reflected signals can be used to reduce motion artifacts. In some embodiments, merging the at least two reflected signals involves applying a matched filter that is based on an ideal period shape to the at least two reflected signals. In some embodiments, merging the at least two reflected signals involves applying a Woody filter to the at least two reflected signals.

Merging the at least two reflected signals can be based on a position of one or more emitters that transmit the one or more electromagnetic signals, relative to the tissue boundary of the subject. For example, if there are two emitters, a first emitter positioned at a first distance from the tissue boundary and a second emitter positioned at a second distance from the tissue boundary, the penetration depth of signals transmitted from the first emitter can be different then the penetration depth of the signals transmitted from the second emitter. Merging signals received by each of the first and second emitter accounts for this difference in position by accounting for the fact that the received physiological signal based on each emitter are substantially the same while the noise is different. Thus, merging the signals yields one physiological signal form which the noise can be extracted.

Merging the at least two reflected signals can be based on a wavelength of the transmitted electromagnetic signals. For example, for emitters that are multi-wavelength, if the wave-length changes, the penetration depth of signals transmitted at a first wavelength can be different then the penetration depth of the signals transmitted at a second wavelength. Merging signals received by each of the first and second emitter accounts for this difference in wavelength by accounting for the fact that the received physiological signal based on each emitter are substantially the same while the noise is different

The method also involves receiving and de-correlating one or more reference signals (e.g., the reference signals as described above in FIG. 1) (Step 330). The one or more reference signals can indicate motion of the subject at the time the at least two reflected signals were detected. The one or more reference signals can be received from one or more accelerometers (e.g., one or more accelerometers as described above in FIG. 1). The one or more reference signals can be de-correlated based on principle component analysis.

In some embodiments, the one or more reference signals are received from MEMS sensors. In some embodiments, the one or more reference signals are received from a gyroscope. In some embodiments, the one or more reference signals are received from an optical reference. The optical reference can be a wavelength that is not absorbed by the red blood cells in the tissue, and thus in the absence of motion, reflects with only a DC component. Once there is motion, the reflections include an AC component, such that the motion can be detected.

In some embodiments, the one or more reference signals are determined by applying a polarization filter to the at least two reflected signals. In these embodiments, only a portion of at least two reflected signals is polarized (e.g., the reflected portion from the tissue boundary is polarized while the reflected portion from the tissue is not polarized). In these embodiments, a portion of the at least two reflected signals that is polarized reflects how much of the at least reflected signals are noise and a portion of the at least two reflected signals that is not polarized reflects how much of the at least reflected signals is the desired signal (e.g., it is possible to determine the physiological parameter from the signal).

The method also involves determining an activity state (Step 335). The activity state can be based on the one or more reference signals. The activity state can be based on a velocity and/or acceleration of the subject as determined from the one or more references signals. In some embodiments, the activity state can be determined, as shown below in Table 1.

TABLE 1

| Activity State | Velocity of the Subject | |
|---|---|---|
| Rest | 0.0 | m/s |
| Beginning of Low Activity | 0.0-0.99 | m/s |
| During Low Activity | 1.0-1.49 | m/s |
| Post Low Activity | 1.5-1.99 | m/s |
| Beginning of High Activity | 2.0-2.49 | m/s |

| | |
|---|---|
| During High Activity | 2.5 and greater |

In some embodiments, the velocity of the subject that corresponds to each activity state is configurable. For example, a lookup table can be used. In some embodiments, the velocity that corresponds to each activity state is adaptive.

The method also involves removing at least a portion of motion noise from the merged signal (Step 340). Removing at least a portion of motion noise from the merged signal can be based on the one or more reference signals to obtain a noise-reduced merged signal. In various embodiments, removing at least a portion of motion noise is based on the one or more reference signals, ambient light surrounding the wearable device, or any combination thereof. In some embodiments, removing at least a portion of motion noise involves determining a linear combination signal based on the one or more reference signals, one or more ambient light signals, and the merged signal. In some embodiments, the linear combination signal is determined as follows:

$$\begin{matrix}( n ) = [ \begin{matrix}x ( n ) \\ x ( n - 1 ) \\ ⋮ \\ x ( n - K )\end{matrix} ] & EQN . 6 \\ ɛ ( n ) = s ( n ) - {X ( n )}^{T} W ( n ) & EQN . 7 \\ g ( n ) = P ( n - 1 ) X ( n ) {( λ + {X}^{T} ( n ) P ( n - 1 ) X ( n ) )}^{- 1} & EQN . 8 \\ P ( n ) = {λ}^{- 1} P ( n - 1 ) - g ( n ) {X}^{T} ( n ) {λ}^{- 1} P ( n - 1 ) & EQN . 9 \\ W ( n ) = W ( n - 1 ) + ɛ ( n ) g ( n ) & EQN . 10\end{matrix}$$

where x(n) is the reference signal, ε(n) is an estimation error, s(n) is the merged signal, W(n) is an adaptive filter weighted sum coefficients, and λ is a forgetting factor.

In some embodiments, a non-linear transformation is applied to the linear combination signal. The non-linear transformation can be based on the one or more reference signals and the one or more ambient light signals. In some embodiments, the non-linear transformation is determined as follows:

$$\begin{matrix}( n ) = f ( [ \begin{matrix}x ( n ) \\ x ( n - 1 ) \\ ⋮ \\ x ( n - K )\end{matrix} ] , s ( n ) ) & EQN . 11 \\ ɛ ( n ) = s ( n ) - {X ( n )}^{T} W ( n ) & EQN . 12 \\ g ( n ) = P ( n - 1 ) X ( n ) {( λ + {X}^{T} ( n ) P ( n - 1 ) X ( n ) )}^{- 1} & EQN . 13 \\ P ( n ) = {λ}^{- 1} P ( n - 1 ) - g ( n ) {X}^{T} ( n ) {λ}^{- 1} P ( n - 1 ) & EQN . 14 \\ W ( n ) = W ( n - 1 ) + ɛ ( n ) g ( n ) & EQN . 15\end{matrix}$$

where x(n) is the reference signal, ε(n) is an estimation error, s(n) is the merged signal, X(n) is a nonlinear combination of x(n) & s(n), W(n) is an adaptive filter weighted sum coefficients and λ is a forgetting factor.

The method also involves determining one or more physiological parameters (Step 345). The one or more physiological parameters can be based on the one or more reference signals, the activity state, the noise-reduced merged signal, or any combination thereof. In some embodiments, the one or more physiological parameters is a heart rate of the subject. In these embodiments, a frequency of the merged signal is determined.

The frequency of the noise-reduced merged signal can be based on a probability using a time-domain, frequency-domain or any combination thereof. In some embodiments, the activity state indicates the portion of the noise-reduced merged signal to consider for the probability determination (e.g., a Kalman filter).

The time-domain estimation can be an instantaneous average interval between local peaks of the merged signal (e.g., a PPG signal). The frequency-domain estimate can be a level of confidence frequency-domain estimate based on a kurtosis of spectral energy of the merged signal (e.g., the PPG signal). The frequency-domain estimation can be a chirp Z transform around the frequency of interest. The frequency of interest can be the frequency with the highest energy level in the chirp Z transform.

The method also involves frequency quality control (Step 350). The frequency quality control can involve assessing an amount of the physiological parameter in the merged signal (e.g., SNR). After extracting the physiological parameter an assessment can be performed done of how much of the merged signal energy represent the physiological parameter. The SNR of the merged signal can be determined as shown below in EQN. 15

$$SNR = \frac{{\int }_{{ω}_{0} - δ}^{{ω}_{0} + δ} S ( ω ) \partial ω}{{\int }_{0}^{π} S ( ω ) \partial ω} \qquad EQN . 15$$

where ω₀ is the physiological parameter, S(ω) is the short time Fourier transform of the merged signal, and δ is accuracy tolerance based on physiological behavior, algorithmic limitations or any combination thereof. The frequency quality control can be used for an analog AGC to assist, for example, in reducing power consumption.

The method also involves controlling a sample rate of the one or more reflected signals, the one or more reference signals, the one or more ambient light signals, or any combination thereof (Step 355). The sampling rate can be based on the frequency of the noise-reduced merged signal. For example, for a physiological parameter of a heart rate, if the heart rate detected is low (e.g., 40-100), the one or more reflected signals and the one or more reference signals, the one or more ambient light signals, or any combination thereof, can be sampled at a rate of 8 hertz. In another example, for a physiological parameter of a heart rate, if the heart rate detected is high (e.g., 120-140), the one or more reflected signals and the one or more reference signals, the ambient light signals, or any combination thereof, can be sampled at a rate of 20 hertz.

As is apparent to one of ordinary skill in the art, determining the one or more physiological parameters can exclude one or more of the steps above described above in method 300. For example, in some embodiments, determining the one or more physiological parameters involves only steps 325, 330, 340 and 345. In another example, determining the one or more physiological parameters involves only steps 325, 330, 335, 340 and 345.

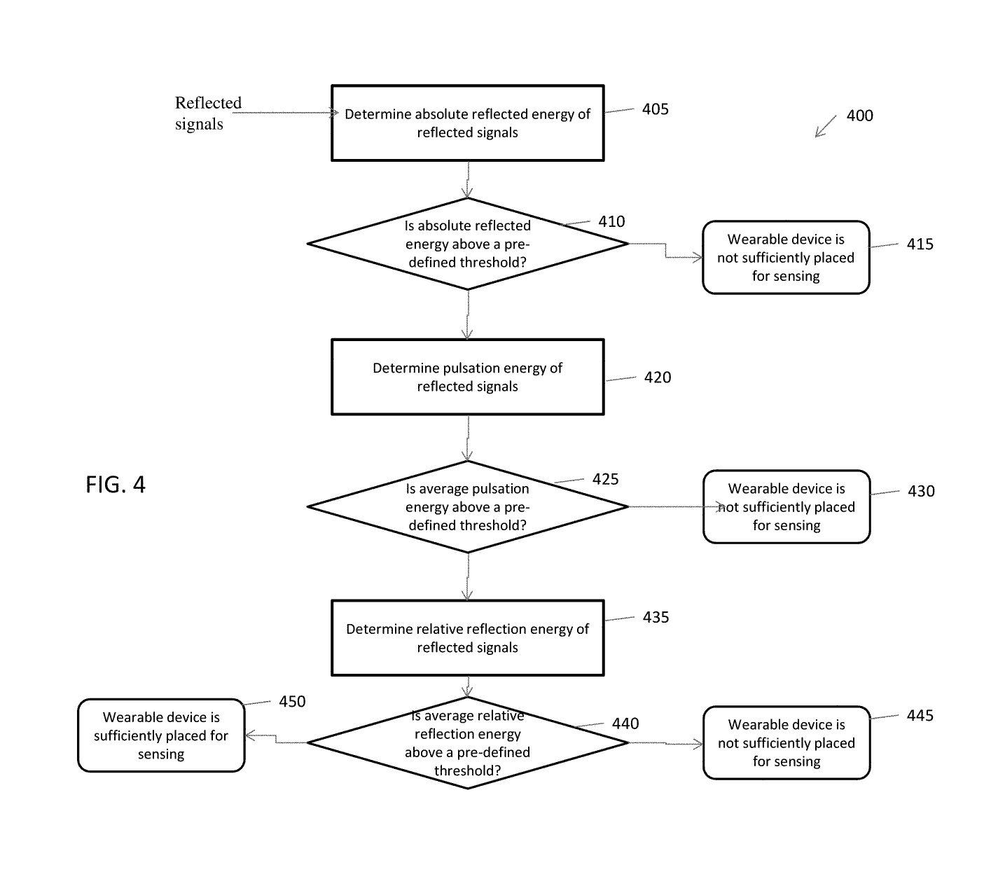

FIG. 4 is a flow diagram of a method 400 for detecting wearable device is sufficiently placed in proximity to a tissue boundary of a subject, according to an illustrative embodiment of the invention. The method involves determining an absolute reflected energy of one or more reflected signals (Step 405). The method also involves determining if the absolute energy is above a pre-defined threshold (Step 410). In some embodiments, the pre-defined threshold is based on intensity and/or wavelength of the emitter. In some embodiments, the pre-defined threshold is based on 15% of the energy of the emitter. In some embodiments, the pre-defined threshold is 10,000.

If the absolute energy is below the pre-defined threshold, then the wearable device is not sufficiently placed for sensing (Step 415). If the absolute reflected energy of the reflected signals is above the pre-defined threshold, then determine pulsation energy of the reflected signals (Step 420).

The method also involves determining if the pulsation energy is above a pre-defined threshold (Step 425). In some embodiments, the pre-defined threshold is based on an ideal period shape.

If the pulsation energy is below the pre-defined threshold, then the wearable device is not sufficiently placed for sensing (Step 430). If the pulsation energy is above the pre-defined threshold, then determine relative reflection energy of the reflected signals (Step 435).

The method also involves determining if the relative reflection energy is above a pre-defined threshold (Step 440). In some embodiments, the pre-defined threshold is based on whether the relative reflection energy is AC or DC.

If the relative reflection energy is below the pre-defined threshold, then the wearable device is not sufficiently placed for sensing (Step 445). If the pulsation energy of the reflected signals is above the pre-defined threshold, then the wearable device is sufficiently placed for sensing (Step 450).

In some embodiments, if the average pulsation energy is above the pre-defined threshold as shown in Step 425, then a conclusion that the wearable device is sufficiently placed for sensing is made, and steps 435 and 440 are skipped.

The above-described computer-implemented methods can be implemented in digital electronic circuitry, in computer hardware, firmware, and/or software. The implementation can be as a computer program product (e.g., a computer program tangibly embodied in an information carrier). The implementation can, for example, be in a machine-readable storage device for execution by, or to control the operation of, data processing apparatus. The implementation can, for example, be a programmable processor, a computer, and/or multiple computers.

A computer program can be written in any form of programming language, including compiled and/or interpreted languages, and the computer program can be deployed in any form, including as a stand-alone program or as a subroutine, element, and/or other unit suitable for use in a computing environment. A computer program can be deployed to be executed on one computer or on multiple computers at one site.

Method steps can be performed by one or more programmable processors executing a computer program to perform functions of the invention by operating on input data and generating output. Method steps can also be performed by an apparatus and can be implemented as special purpose logic circuitry. The circuitry can, for example, be a FPGA (field programmable gate array) and/or an ASIC (application-specific integrated circuit). Modules, subroutines, and software agents can refer to portions of the computer program, the processor, the special circuitry, software, and/or hardware that implement that functionality.

Processors suitable for the execution of a computer program include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data. Generally, a computer can be operatively coupled to receive data from and/or transfer data to one or more mass storage devices for storing data (e.g., magnetic, magneto-optical disks, or optical disks).

Data transmission and instructions can also occur over a communications network. Information carriers suitable for embodying computer program instructions and data include all forms of non-volatile memory, including by way of example semiconductor memory devices. The information carriers can, for example, be EPROM, EEPROM, flash memory devices, magnetic disks, internal hard disks, removable disks, magneto-optical disks, CD-ROM, and/or DVD-ROM disks. The processor and the memory can be supplemented by, and/or incorporated in special purpose logic circuitry.

To provide for interaction with a user, the above described techniques can be implemented on a computer having a display device, a transmitting device, and/or a computing device. The display device can be, for example, a cathode ray tube (CRT) and/or a liquid crystal display (LCD) monitor. The interaction with a user can be, for example, a display of information to the user and a keyboard and a pointing device (e.g., a mouse or a trackball) by which the user can provide input to the computer (e.g., interact with a user interface element). Other kinds of devices can be used to provide for interaction with a user. Other devices can be, for example, feedback provided to the user in any form of sensory feedback (e.g., visual feedback, auditory feedback, or tactile feedback). Input from the user can be, for example, received in any form, including acoustic, speech, and/or tactile input.

The computing device can include, for example, a computer, a computer with a browser device, a telephone, an IP phone, a mobile device (e.g., cellular phone, personal digital assistant (PDA) device, laptop computer, electronic mail device), and/or other communication devices. The computing device can be, for example, one or more computer servers. The computer servers can be, for example, part of a server farm. The browser device includes, for example, a computer (e.g., desktop computer, laptop computer, and tablet) with a World Wide Web browser (e.g., Microsoft® Internet Explorer® available from Microsoft Corporation, Chrome available from Google, Mozilla® Firefox available from Mozilla Corporation, Safari available from Apple). The mobile computing device includes, for example, a personal digital assistant (PDA).

Website and/or web pages can be provided, for example, through a network (e.g., Internet) using a web server. The web server can be, for example, a computer with a server module (e.g., Microsoft® Internet Information Services available from Microsoft Corporation, Apache Web Server available from Apache Software Foundation, Apache Tomcat Web Server available from Apache Software Foundation).

The storage module can be, for example, a random access memory (RAM) module, a read only memory (ROM) module, a computer hard drive, a memory card (e.g., universal serial bus (USB) flash drive, a secure digital (SD) flash card), a floppy disk, and/or any other data storage device. Information stored on a storage module can be maintained, for example, in a database (e.g., relational database system, flat database system) and/or any other logical information storage mechanism.

The above-described techniques can be implemented in a distributed computing system that includes a back-end component. The back-end component can, for example, be a data server, a middleware component, and/or an application server. The above described techniques can be implemented in a distributing computing system that includes a front-end component. The front-end component can, for example, be a client computer having a graphical user interface, a Web browser through which a user can interact with an example implementation, and/or other graphical user interfaces for a transmitting device. The components of the system can be interconnected by any form or medium of digital data communication (e.g., a communication network). Examples of communication networks include a local area network (LAN), a wide area network (WAN), the Internet, wired networks, and/or wireless networks.

The system can include clients and servers. A client and a server are generally remote from each other and typically interact through a communication network. The relationship of client and server arises by virtue of computer programs running on the respective computers and having a client-server relationship to each other.

The above described networks can be implemented in a packet-based network, a circuit-based network, and/or a combination of a packet-based network and a circuit-based network. Packet-based networks can include, for example, the Internet, a carrier internet protocol (IP) network (e.g., local area network (LAN), wide area network (WAN), campus area network (CAN), metropolitan area network (MAN), home area network (HAN), a private IP network, an IP private branch exchange (IPBX), a wireless network (e.g., radio access network (RAN), 802.11 network, 802.16 network, general packet radio service (GPRS) network, HiperLAN), and/or other packet-based networks. Circuit-based networks can include, for example, the public switched telephone network (PSTN), a private branch exchange (PBX), a wireless network (e.g., RAN, Bluetooth®, code-division multiple access (CDMA) network, time division multiple access (TDMA) network, global system for mobile communications (GSM) network), and/or other circuit-based networks.

Comprise, include, and/or plural forms of each are open ended and include the listed parts and can include additional parts that are not listed. And/or is open ended and includes one or more of the listed parts and combinations of the listed parts.

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

What is claimed is:

1. A method of detecting one or more physiological parameters, the method comprising:
transmitting one or more electromagnetic signals from a wearable device to a tissue boundary of a subject;
receiving at least two reflected signals based on reflections from tissue of the one or more electromagnetic signals;
receiving a reference signal based on motion of the subject;
merging the at least two reflected signals based on a position of one or more receivers that receive the at least two reflected signals to create a merged signal;
removing at least a portion of motion noise from the merged signal based on the reference signal to obtain a noise-reduced merged signal; and
determining the one or more physiological parameters based on the noise-reduced merged signal.

2. The method of claim 1 wherein the reference signal is one or more three-dimensional acceleration signals.

3. The method of claim 1 wherein determining the one or more physiological parameters further comprises:
receiving acceleration signals from an accelerometer coupled to the subject;
determining an activity state of the subject based on the acceleration signals; and
determining a frequency for the one or more physiological parameters based on the activity state of the subject.

4. The method of claim 1 wherein removing at least a portion of motion noise from the merged signal further comprises:
receiving ambient light signals from a light sensor coupled to the subject;
determining a linear combination signal based on the reference signal, the ambient light signals and the merged signal;
determining a non-linear transformation of the linear combination signal based on the reference signal and the ambient light signals; and
applying the non-linear transformation to obtain the noise-reduced merged signal.

5. The method of claim 1 wherein the one or more physiological parameters comprises a heart rate.

6. The method of claim 1 further comprising de-correlating the reference signal based on principle component analysis of the reference signal.

7. The method of claim 1 wherein determining the one or more physiological parameters further comprises sampling the at least two reflected signals based on one or more previously determined physiological parameters of the subject.

8. The method of claim 1 wherein the one or more electromagnetic signals comprise a wavelength, amplitude or both that is based on tissue color of the subject, ambient light, or any combination thereof.

9. The method of claim 1 wherein the one or more electromagnetic signals are transmitted with a wavelength of green light or infrared light.

10. The method of claim 1 further comprising increasing or decreasing an amplitude of the one or more electromagnetic signals based on ambient light surrounding the subject.

11. The method of claim 1 further comprising normalizing the at least two reflected signals such that each of the at least two reflected signals have a variance below a predetermined variance threshold.

12. The method of claim 1 wherein the one or more electromagnetic signals are generated by one or more light emitting diodes.

13. The method of claim 1 wherein the at least two reflected signals are received by one or more photo sensors.

14. The method of claim 1 wherein the at least two reflected signals are photoplethysmogram (PPG) signals.

15. The method of claim 1 wherein determining the one or more physiological parameters further comprises:
comparing the one or more physiological parameters to previously determined one or more physiological parameters, respectively; and
for each of the one or more physiological parameters, if the comparison is greater than a predetermined threshold, discarding the respective determined one or more physiological parameters.

16. The method of claim 1 further comprising:
receiving by the wearable device audio signals; and
processing the audio signals on a processor within the wearable device, the processor being the same processor that the one or more physiological parameters are processed on.

17. The method of claim 1 wherein the wearable device is an earbud, a watch, a chest strap, or any combination thereof.

18. A system for detecting one or more physiological parameters, the method comprising:
one or more emitters to transmit one or more electromagnetic signals from a wearable device to a tissue boundary of a subject;
one or more detectors to receive at least two reflected signals based on reflections from the tissue boundary of the one or more electromagnetic signals;
one or more accelerometers to receive a reference signal based on motion of the subject;
one or more processor configured to:
a) merge the at least two reflected signals based on a position of one or more receivers that receive the at least two reflected signals to create a merged signal;
b) remove at least a portion of motion noise from the merged signal based on the reference signal to obtain a noise-reduced merged signal; and
c) determine the one or more physiological parameters based on the noise-reduced merged signal.

19. A method of detecting when an earbud is sufficiently inserted into an ear, the method comprising:
transmitting a first electromagnetic energy with a wavelength from the earbud;
receiving reflected electromagnetic energy by the earbud;
determining an absolute reflection energy based on the reflected electromagnetic energy;
determining a pulsation energy based on the reflected electromagnetic energy; and
determining whether the earbud is sufficiently inserted into the ear based on the absolute reflection energy and the pulsation reflection energy.

20. The method of claim 19 further comprising:
determining a relative reflection energy gradient based on the reflected electromagnetic energy.
